(19) United States
(12) Patent Application Publication
(10) Pub. No.: US 2010/0129305 A1
(43) Pub. Date: May 27, 2010

(54) Compositions Containing Extracts From Radish

(76) Inventors: Wilson A. Lee, Hauppauge, NY (US); Georgena Moreira Keupp, Bay Shore, NY (US); Hernando Brieva, Manalapan, NJ (US); Matthew Ronald Warren, South Huntington, NY (US)

Correspondence Address: THE ESTEE LAUDER COS, INC, 155 PINELAWN ROAD, STE 345 S, MELVILLE, NY, 11747, US

(21) Appl. No.: 12/615,747

(22) Filed: Nov. 10, 2009

Related U.S. Application Data
(60) Provisional application No. 61/116,880, filed on Nov. 21, 2008.

(51) Int. Cl. A61K 8/97; A61Q 1/10; A61Q 19/00

(52) U.S. Cl. 424/63; 424/755

(57) ABSTRACT

A topical composition comprising an ingredient that is obtained by fermentation of Raphanus Sativus roots by the organism Leuconostoc or Lactobacillus.

### CROSS-REFERENCE TO RELATED APPLICATIONS

This application claims priority of U.S. Provisional Patent Application Ser. No. 61/116,880, filed on Nov. 21, 2008.

### TECHNICAL FIELD

The invention is in the field of topical compositions and methods for treating keratinous surfaces for improvement.

### BACKGROUND OF THE INVENTION

Cosmetics companies are always searching for improved ingredients that exhibit anti-bacterial properties and thus a preservative function. Cosmetics must have excellent preservative systems to ensure that they do not become contaminated with bacteria while being stored and used. The number of preservatives that are suitable for cosmetics are limited. Further, certain types of cosmetics like mascara are notoriously difficult to preserve. For that reason new preservatives, preferably those that are naturally derived and exhibit multiple functionalities are of great interest. More specifically, naturally occurring materials that not only have anti-bacterial properties but exhibit stability in cosmetic formulations and provide some of the other advantages of botanical extracts such as anti-oxidant properties, anti-acne properties, or anti-inflammatory properties, are very desirable. That way the preservative will have multiple functionalities and enable use of fewer raw materials in cosmetic formulations. This is always desirable from the cost point of view.

It has been discovered that an extract from radish roots, when fermented, provides excellent preservative activity, particularly in hard to preserve compositions. In addition, the extract is stable in cosmetic formulations and can be used alone without any additional preservatives, or with reduced levels of standard preservatives. The extract also has other beneficial properties such as anti-oxidant, anti-inflammatory, and anti-acnegenic activity.

It is an object of the invention to provide a cosmetic composition comprising a fermented extract of Raphanus Sativus or radish root.

It is a further object of the invention to provide mascara and skin care compositions containing at least one fermented extract of Raphanus Sativus or radish root.

It is a further object of the invention to provide cosmetic compositions comprising a naturally occurring ingredient stable in cosmetic formulations and having multiple functionalities selected from preservative, antioxidant, anti-acne, anti-inflammatory, and the like.

### SUMMARY OF THE INVENTION

The invention is directed to topical compositions comprising an ingredient that is obtained by fermentation of Raphanus Sativus roots by the organism Leuconostoc or Lactobacillus.

The invention is further directed to a mascara composition comprising an ingredient that is obtained by fermentation of Raphanus Sativus roots by the organism Leuconostoc or Lactobacillus, and further comprising from about 0.1-95% water, from about 0.1-95% oil, from about 0.1 to 45% of a film former, and from about 0.1-30% pigments.

The invention is further directed to a skin treatment composition in the form of a water and oil emulsion comprising an ingredient that is obtained by fermentation of Raphanus Sativus roots by the organism Leuconostoc or Lactobacillus, water, at least one silicone oil, at least one botanical extract, and at least one humectant.

The invention is further directed to an aqueous based skin treatment composition comprising an ingredient that is obtained by fermentation of Raphanus Sativus roots by the organism Leuconostoc or Lactobacillus, from about 0.1-99% water, about 0.001-15% of at least one botanical extract, and from about 0.1-45% of an aqueous phase thickening agent.

The invention is further directed to an oil in water emulsion skin care composition comprising from about 0.1 to 99% oil, from about 0.1-99% water, wherein the oil phase comprises at least one silicone selected from cyclomethicone, dimethicone, methyl trimethicone, PEG-10 dimethicone, or mixtures thereof; and the composition further comprises at least one organosiloxane elastomer in an amount of about 0.1 to 50%; and about 0.01-20% of at least one humectant which is butylene glycol, propylene glycol, hexylene glycol, glycerin, or mixtures thereof

### DETAILED DESCRIPTION

The compositions of the invention are leave on compositions, meaning that they are applied to the skin or lips and left on as opposed to being applied and washed off (e.g. shampoos, skin cleansers, etc.). The compositions of the invention are topical cosmetic or pharmaceutical compositions. They may be in the anhydrous or aqueous gel, solution or serum form. They may also be in the emulsion form, that is, water in oil or oil in water emulsions. If in the anhydrous form, the composition generally comprises from about 0.1 95% oil, and optionally other ingredients such as powders, sunscreens, and the like, with all percentages meaning percent by weight unless otherwise indicated. If the aqueous gel, solution, or serum form the composition comprises from about 1-99% water, and optionally other ingredients. If in the emulsion form, the composition may comprise from about 0.1 to 99%, preferably from about 5-95%, more preferably from about 7 to 90% water and from about 0.1 to 99%, preferably from about 5-95%, more preferably from about 7 to 90% oil.

### I. The Radish Root Ferment

The composition of the invention comprises at least one radish root ferment. Suggested ranges are from about 0.001 to 75%, preferably from about 0.05 to 70%, more preferably from about 0.1 to 65%. The preferred radish root ferment may be sourced from Mercordi Animal Care and is identified by the trademark Kimchistoc® and has the INCI name Leuconostoc/Radish Root Ferment Filtrate, which is described as the filtrate of the product obtained by the fermentation of Raphanus Sativus roots by the microorganism Leuconostoc. The radish root may also be fermented with other organisms such as Lactobacillus. The ingredient may also be purchased from Active Micro Systems under the trademark AMS Leucidal Liquid, or from Active Concepts LLC under the trademark Leucidal.

### II. The Leave on Compositions

The compositions of the invention are applied to keratinous surface such as skin, lips, or eyelashes. They may be cosmetic or pharmaceutical compositions. Types of compositions include mascara, blush, eye shadow, eyeliner, skin cream, skin lotion, skin treatment serum, foundation makeup, lipstick, lip gloss, and the like.

A. Mascara

The composition of the invention may be in the form of a mascara for application to the eyelashes to provide color. Mascaras may be anhydrous or in the water in oil or oil in water emulsion form. Anhydrous mascaras comprise from about 0.1-95% oil, from about 0.1-35% pigments, and from about 0.1-40% film former. Emulsion mascaras comprise from about 1-95% water, from about 0.1-95% oil, from about 0.1-35% pigments and from about 0.1-40% film former, and, optionally, other ingredients.
1. Oils

The mascara will generally contain one or more oils which may be volatile or non-volatile. The term “oil” refers to an ingredient that is pourable at room temperature (e.g. 25° C.) The term “volatile” means that the oil has a vapor pressure of greater than about 2 mm. of mercury at 20° C. Volatile oils may include silicones or paraffinic hydrocarbons. If present such volatile oils may range from about 0.1 to 50%. Examples of volatile silicones include linear silicones such as hexamethyldisiloxane (0.5 centistokes (cs)), octamethyltrisiloxane (1.0 cs), decamethyltetrasiloxane (1.5 cs), dodecamethylpentasiloxane (2.0 cs); or cyclic volatile silicones generally referred to as “cyclomethicone”, which may be octamethylcyclotetrasiloxane, decamethylcyclopentasiloxane, dodecamethylcyclohexasiloxane, and so on. Examples of volatile paraffinic hydrocarbons include isohexadecane, isododecane, C9-11 isoparaffins, and the like.

Nonvolatile oils may include silicones, hydrocarbons, or esters. The term “nonvolatile” means that the oil has a vapor pressure of less than about 2 mm. of mercury at 20° C. Examples of nonvolatile silicones include dimethicone or diethicone; phenyl-substituted silicones such as phenyl trimethicone, diphenyl dimethicone, phenyl dimethicone, trimethylsiloxyphenyl dimethicone, phenethyl dimethicone; and the like; C2-14 alkyl substituted silicones such as cetyl dimethicone, hexyl dimethicone, lauryl dimethicone; or fluorinated silicones such as perfluorononyl dimethicone, trifluoropropyl dimethicone; and the like.

Examples of non-volatile hydrocarbons include hydrocarbons having from 10 to 40 carbon atoms in the linear or branched form such as C10-40 isoparaffins, C12-20 isparaffins; or olefins having from about 18 to 54 carbon atoms, for example C18-26 olefin, C20-24 olefin, C26-54 olefin; polybutene, polyisobutene, polydecene, or hydrogenated derivatives thereof such as hydrogenated polyisobutene; isoeicosane; squalane; squalene; and so on.

Examples of esters include those formed by the reaction of mono-, di-, or polyhydric C1-10 alcohols with carboxylic acids having from about 1 to 40 carbon atoms, preferably fatty C6-22 carboxylic acids. Preferred alcohols include ethanol, propanol, butanol, hexanol, glycerin, and so on. Preferred carboxylic acids include myristic, stearic, isostearic, palmitic, behenic, and so on. One particularly preferred ester is formed by the reaction of a polyhydric alcohol with fatty carboxylic acids, more specification glycerin and stearic acid, to provide glyceryl stearate, diglyceryl diisostearate, glyceryl triisostearate, and so on. Most preferred is glyceryl stearate.
2. Film Formers

The mascara composition comprises at least one film former which is capable of forming a film when applied to the lashes in the mascara form. Suggested ranges of film former are from about 0.1 to 45%, preferably from about 0.5 to 40%, more preferably from about 0.5 to 30%. Film formers may be synthetic or natural polymers. They may be water soluble or oil soluble. They may be in the form of particles in aqueous dispersion, or solubilized or dispersed in the lipophilic phase of the composition. Examples of film forming polymers include polyurethanes either in lipophilic form or in the form of particles in aqueous dispersion; copolymers from ethylenically unsaturated monomers, for example, homo- or copolymers of acrylates, ammonium acrylates, styrene, acrylamides, methacrylates, vinyl acetate, vinyl pyrrolidone; or siloxy silicate polymers such as trimethylsiloxysilicate, polymethylsilsesquioxane; or silicone gums which are general dimethicone or dimethiconol having a degree of polymerization ranging from about 100,000 to 100 million cs. Examples of such film forming polymers include, but are not limited to, acrylates copolymer, polyurethane, acrylamide/ammonium acrylate copolymer, acrylamides copolymer, acrylates/acrylamide copolymer, acrylates/ammonium methacrylate copolymer, acrylates/octylacrylamide copolymer, PVP, PVA, PVP/VA copolymer, acrylates/VA copolymer, acrylates/VP copolymer, ammonium styrene/acrylates copolymer, and so on. Also suitable are naturally occurring polymers such as shellac or cellulose. Preferred is where the film former is shellac in aqueous dispersion which comprises from about 10-70% water, 1-40% shellac, and optionally from about 0.1-40% of a monohydric alcohol (preferably isopropanol or ethanol) and from about 0.0001 to 10% of a neutralizer which may be ammonium hydroxide. Also preferred is where the film former comprises a vinyl pyrrolidone homo- or copolymer such as PVP, PVP/VA copolymer and so on.
3. Structuring Agents

Preferred mascara compositions contain at least one structuring agent which increases the viscosity or thickens the composition. Suggested ranges are from about 0.1-70%, preferably from about 0.5-65%, more preferably from about 1-60%. Structuring agents may be natural or synthetic waxes, polymeric materials that increase viscosity, and the like. Examples of waxes include polyethylene, polypropylene, beeswax, beeswax modified by reacting with polyethylene glycol, such as PEG-8 beeswax, PEG-10 beeswax, carnauba, ceresin, microcrystalline, or fatty alcohols such as stearyl, behenyl; or fatty acids such as stearic acid, behenic acid, isostearic acid, and so on. Also suitable as structuring agents are polyamides or silicone polyamides. Examples include those sold by Arizona Chemical under the trademarks Sylvaclear® and Uniclear®, including those having the INCI names ethylenediamine/stearyl dimer tallate copolymer or ethylenediamine/stearyl dimer dilinolate copolymer, ethylenediamine/dimer tallate copolymer bis-hydrogenated tallow amide; Polyamide-3; ethylenediamine/hydrogenated dimer dilinoleate copolymer bis-di-C14-18 alkyl amide; Polyamide-4; bis-stearyl ethylenediamine/neopentyl glycol/stearyl hydrogenated dimer dilinoleate copolymer and the like. Silicone waxes may also be used including but not limited to stearyl dimethicone, behenyl dimethicone, behenoxy dimethicone, stearoxy dimethicone, and so on. Also suitable as structuring agents are N-acyl amino acids or esters or amides thereof; 12-hydroxystearic acid or esters or amides thereof; fatty acid esters of di- or trifunctional alcohol dimers; or alkylamides of di- or tricarboxylic acids. Examples include stearamide MEA-stearate, N-acyl glutamic acid diamide, and so on.

Also suitable as structuring agents are naturally occurring ingredients that increase viscosity such as gums, including but not limited to Acacia Senegal gum, Veegum (magnesium aluminum silicate), biosaccharide gum, Boswellia Serrata gum, Cassia gum, cellulose gum, Gellan gum, xanthan gum, algin, agarose, cellulose, hydroxyethylcellulose, and the like.

Silicone elastomers are also suitable structuring agents and include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.
4. Emulsifiers

The mascara composition preferably comprises at least one emulsifier or surfactant. Suggested ranges are from about 0.01-40%, preferably from about 0.05-35%, more preferably from about 0.1-25%.

Suitable emulsifiers may be silicone based emulsifiers or surfactants including linear or crosslinked polyoxyalkylene substituted organosiloxanes or alkyl substituted polyoxyalkylene organosiloxanes. Examples include those having the generic name dimethicone copolyol, cetyl dimethicone copolyol, and so on. Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Coming 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21 , KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable are ethoxylated propoxylated derivatives of C6-30 saturated or unsaturated fatty acids, for example, Di-PPG-2 myreth-10 adipate, Di-PPG-2 Ceteth-4 adipate, Di-PPG Myristyl Ether Adipate,

Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Most preferred for use in mascaras are organic emulsifers that are the reaction product of polyethylene glycol with fatty acids, e.g. PEG1-200 with stearic, isostearic, myristic, behenic, cetearic, acids and so on. Particulately preferred is where the emulsifier comprises the reaction product of polyethylene glycol and stearic acid, e.g. PEG-100 stearate. Also suitable are derivatives of sorbitan such as Polysorbates; sorbitan esterified with stearic acid, e.g. sorbitan tristearate, and so on.
5. Particulates

The mascara composition comprises particulates, either in the from of pigments, powders or mixtures. Suitable pigments include inorganic pigments or iron oxides that are red, yellow, or black. Also suitable are organic pigments that are FD&C or D&C colors or Lakes thereof such as yellows, reds, blues, etc. Suitable powders include titanium dioxide, mica, bismuth oxychloride, titanated mica, PTFE, silica, bentonite, kaolin, talc, and the like. Suggested ranges of pigments are from about 0.01-45%, preferably from about 0.05-35%. Suggested ranges of powders are from about 0.01-10%.

The mascara composition is preferably in the oil in water emulsion form. The composition may contain other ingredients, including but not limited to humectants, preservatives, pH adjusters, and so on. Preferred mascara compositions are substantially free of paraben preservatives, and may contain the radish root ferment as a preservative system for the mascara either alone or in combination with other non-paraben preservatives, e.g. phenoxyethanol.

B. Skin Creams, Lotions, Serums

The radish root ferment may be used in skin care products such as skin creams, lotions, solutions, or serums. These compositions may be in the emulsion form, that is water in oil or oil in water. Alternatively, they may be in the aqueous gel or serum form. Water in oil or oil in water emulsions may comprise from about 1 to 99% water and from about 01 to 99% oil. Aqueous gels or serums generally comprise from about 5-100% water in addition to the radish root ferment.
1. Oils

The skin care compositions may also contain one or more oils of the type and in the amounts set forth with respect to the mascara composition.
2. Structuring Agents

The skin care compositions may further comprise one or more structuring agents of the type and in the amounts set forth with respect to the mascara composition.
3. Emulsifiers or Surfactants

The skin care compositions may further comprise one or more surfactants as set forth above with respect to the mascara composition and in the same general percentage ranges.
4. Aqueous Phase Thickeners

The skin care composition may also contain one or more aqueous phase thickeners. If present, suggested ranges are from about 0.1-30%, preferably from about 0.5-25%, more preferably from about 0.5-20%. Suitable thickeners include acrylic polymeric thickeners comprised of monomers of acrylic acid, methacrylic acid, or their simple C₁₋₂₂ alkyl esters. Acrylic polymer solutions include those sold by Seppic, Inc., under the tradename Capigel having the CTFA name Acrylates Copolymer.

Also suitable are acrylic polymeric thickeners that are copolymers of acrylic acid, methacrylic acid or their C₁₋₂₂ alkyl esters further copolymerized with one or more fatty alkoxylated alcohols. Examples of such thickening agents include acrylates/steareth-20 methacrylate copolymer, which is sold by Rohm & Haas under the tradename Acculyn 22, or acrylates/steareth-20/methacrylate crosspolymer which may be purchased from Rohm & Haas under the tradename Acculyn 88.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specificially a vinyl carboxylic acid such as acrylic acid, methacrylic acid or mixtures thereof, and where the allyl ether unit containing a fatty chain corresponds to the monomer of formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C 18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth- 10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames.

Particularly suitable as the aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

Also suitable as aqueous phase thickening agents are various types of polysaccharides, such as xanthan gum, cellulose, dextrin, cyclodextrin, hydroxyethylcellulose, acacia gum, and the like.
5. Humectants

The composition may also contain one or more humectants. If present suggested ranges are from about 0.1 to 25%. Humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, trehalose, and so on. Also suitable is urea or sugar derivatives, e.g. ethylhexylglycerin. In one preferred embodiment, the humectants used in the composition of the invention are C₁₋₆, preferably C₂₋₄ alkylene glycols, most particularly butylene glycol.
6. Botanical Extracts

A variety of botanical extracts, oils, or butters that may exhibit anti-oxidant activity or have other functional properties may also be used in the skin care compositions, including but not limited to those obtained from roots, leaves, flowers, stalks or other parts of plants such as Simmondsia Chinensis, Camellia Sinensis, Coffee Arabica, Betula Alba, Zea Mays, Siegesbeckia Orientalis, Helianthus Annus, Hordeum Vulgare, Cucumis Sativus, Carthamus Tinctorius, Mangifera Indicia, Garcinia Indica, Anthemus Nobilis, Arabidopsis Thaliana, Myrtus Communis, Apium Graveolens (Celery), Arabidopsis Thaliana, Padina Pavonica, Beta Vulgaris, Betula Alba, Butyrosperum Parkii, Garcinia Mangostana, Racemusus, Platycodon, Emblica Officinalis, Criste Marine, Lavande Papillon, Polygonum Cuspidatum, Laminaria Japonica, Fucus Vesiculosis, Borago Officinalis, Phyllanthus Emblica, Inonotus Obliquus, Calophyllum Inophyllum, Scutellaria Baicalensi), Boswellia Serrata, Boswellia Bhau-dajiana, Boswellia Frereana, Boswellia Papyrifera, Sudanese Boswellia Sacra, Boswellia Carteri, Commiphora Incisa, Commiphora Myrrha, Commiphora Abyssinica, Commiphora Erthraea, Commiphora Molmol, Bursera Microphylla; Nidularium Procerumt, Curcuma Longa, Macrycystis Pyrifera, Pleurotus Ostreatus, Hypsizygus Ulmarius, Cladosiphon Okamuranus, Acalypha Wilkesiana, Acanthopanax Gracilistylus, Allium Sativum, Ananus Comosus, Cissainpelos Sympodialis, Coriolus Versicolor, Echinacea Purpurea, Frondasv, Harpagophytum Procumbens, Panax Ginseng, Polygala Tenuifblia, Pork, Cocos, Silybum Marianum, Smilax Glabra, Tinospora Cordifolia, Uncaria Tomentosa, Withania Somnifera, Echinancea, Viscum Album, Capparis Moonii, Capsella Brusa Pastoris, Doliocarpus Verruculosus, Kaempferia Galanga, Sauropus Androgynus, Tetracapidium Conophorum, Pinus Pinaster, Vitis Vinefera, Pluchea Indica, Viola Hondoensis, Triphala Chebula, Citri Reticulatae, Tepescohuite, Mimosa Pudica, Silymarin, Eucommia, Menyanthes Trifoliata, Calluna Vulgaris, Rosa Canina, Polyporus Umbellatus, Chamomilla Recutita, Pygeum Africanum, Actina Boswellia, Soft Pygeurm Capsicum Annum, Carpinus Laxiflora, Parinus Tschonoskii, Castanopsis Cuspidata, Selaginella Tamariscina, Rosmarinus Officinalis, Cayaponia Tayuya, Celosia, Cristata, Cercis Chinensis, Haplophyllum hispanicum, Scutellaria Rivularis, Centauriumt, Polygonum Cuspidatum, Nigella Sativa, Rhodiola Rosea, Anemarrhena asphodeloides, Zhi Mu, Uncaria Tomentosa Cereus Granidflora, Chaenomeles Sinensis, Lyngbya, Viapure Poria, Polyporus Umbellatus, Chrysanthellum Indicum, Tridentata marginate, Paeonia Alb flora, Saussurea Costus, Saussurea Lappa, Magnolia Officianalis, Echineacea Pallida and those set forth on pages 2755-2757 of the C.T.F.A. International Cosmetic Ingredient Dictionary and Handbook, Eleventh Edition, 2006, which is hereby incorporated by reference in its entirety.
7. Peptides

The composition may also contain one or more peptides with skin beneficial properties. If present, such peptides may range from about 0.01-25%, preferably from about 0.01-10%, more preferably from about 0.01-8%. Suitable peptides include, but are not limited to acetyl hexapeptides 3, 4, 7, 8, 20, 22, or 24; palmitoyl pentapeptides 4 or 5; Pentapeptides 1-19; Tetrapeptides 1-16; Tripeptides 1-29; Palmitoyl tripeptides; Palmitoyl Tetrapeptides; Oligopeptides 1-42; Nonapeptides 1-4; Octapeptides 1-5; and the like. Particularly preferred is Acetyl Hexapeptide-8 (also referred to as Acetyl Hexapeptide-3) sold under the tradename Argireline® by Lipotec S.A.; or Palmitoyl Pentapeptide-4 sold under the tradename Matrixyl® by Sederma. Also suitable are peptides sold under the tradename Vitazyme, such as Vitazyme A Plus which is a mixture of retinol palmitate and carrot polypeptide; Vitazyme B1 which is a mixture of thiamine and yeast polypeptide; Vitazyme B3, which is a mixture of niacinamide and yeast polypeptide; Vitazyme B5 which is a mixture of pantothenic acid and yeast polypeptide; Vitazyme B12 which is a mixture of cyancobalamin and yeast polypeptide; Vitazyme B Complex, which is a mixture of biotin, folic acid, cyancobalamin, niacinamide, pantothenic acid, pyridoxine, riboflavin, thiamine, and yeast polypeptides; Vitazyme C which is a mixture of ascorbic acid, citrus sinensis, citrus limon, and citrus aurantifolia polypeptides; Vitazyme D which is a mixture of cholecalciferol and yeast polypeptides; Vitazyme E, which is a mixture of tocopherol and wheat polypeptides; Vitazyme Retinol which is a mixture of retinol and saccharomyces polypeptide.

The compositions may contain other ingredients including but not limited to preservatives, sunscreens, and so on.

The invention will be further described in connection with the following examples which are set forth for purposes of illustration only.

### EXAMPLE 1

A mascara composition was prepared as follows:

| Ingredient | % by weight |
|---|---|
| Water/PVP/Black iron oxides | 20.00 |
| Methyl trimethicone | 5.75 |
| Leuconostoc/radish root ferment | 2.00 |
| Polyurethane-1 | 13.00 |
| Water | QS |
| Ammonium hydroxide | 0.25 |
| Magnesium aluminum silicate | 0.15 |
| Hydrolyzed jojoba protein | 0.01 |
| Silk amino acids | 0.01 |
| Coffee extract | 0.01 |
| PTFE | 0.01 |
| Pantothenic acid polypeptide | 0.01 |
| Tocopheryl acetate | 0.01 |
| Disodium EDTA | 0.05 |
| Hydroxyethylcellulose | 0.10 |
| Acacia Senegal gum | 0.25 |
| Aminomethyl propanediol | 0.50 |
| Isododecane | 11.75 |
| Polysorbate 20 | 1.25 |
| Silica | 0.50 |
| Kaolin | 2.00 |
| Ethylenediamine/stearyl dimer tallate copolymer/stearic | 21.00 |
| acid/stearamide MEA-stearate/sorbitan tristearate/glyceryl | |
| stearate/PEG-100 stearate | |

The composition was prepared by combining the oil and water phases separately, then mixing to form an oil in water emulsion.

### EXAMPLE 2

Additional mascara compositions were prepared as follows:

| | % by weight | | | |
|---|---|---|---|---|
| Ingredient | 1 | 2 | 3 | 4 |
| Water | QS | QS | QS | QS |
| Ethylenediamine/stearyl dimer | | | 21.00 | 21.00 |
| tallate copolymer/stearic | | | | |
| acid/stearamide MEA- | | | | |
| stearate/sorbitan | | | | |
| tristearate/glyceryl | | | | |
| stearate/PEG-100 stearate | | | | |
| Isododecane | | 16.00 | 11.75 | 11.75 |
| Cyclopentasiloxane | | | 5.75 | 5.75 |
| Water/shellac/isopropyl | | 13.00 | 13.00 | 13.00 |
| alcohol/ammonium hydroxide | | | | |
| Black iron oxides/PEG-9 | 7.00 | | | |
| dimethicone | | | | |
| Silica/black iron | | | 20.00 | 20.00 |
| oxides/kaolin/PVP/water | | | | |
| Black iron oxides | | 6.00 | | |
| Acacia Senegal gum | | 0.25 | 0.25 | 0.25 |
| Stearic acid | 7.00 | 4.50 | | |
| Polypropylene | | 3.25 | | |
| Stearamide MEA Stearate | | 3.00 | | |
| Glyceryl stearate | 6.00 | | | |
| PEG-8 beeswax | 4.80 | | | |
| PEG-100 stearate | | 0.50 | | |
| Polysorbate-20 | | 0.20 | 1.25 | 1.25 |
| Paraffin wax | 4.80 | | | |
| Carnauba | 3.80 | | | |
| Microcrystalline wax | | 1.50 | | |
| Sorbitan tristearate | | 1.00 | | |
| Kaolin | 2.00 | 5.00 | 2.00 | 2.00 |
| Mica | 2.00 | | | |
| Polyisobutene | 2.00 | 6.50 | | |
| Aminomethyl propanediol | 1.64 | 0.50 | 0.50 | 0.50 |
| Bentonite | 1.25 | | | |
| PVP | 1.25 | | | |
| Butylene glycol | 1.00 | | | |
| Leuconostoc/radish root | 1.00 | 1.00 | 2.00 | 1.00 |
| ferment | | | | |
| Green tea extract | 1.00 | | | |
| Silica | 1.00 | | 0.50 | 0.50 |
| Phenoxyethanol/caprylyl | 0.70 | | | |
| glycol/potassium | | | | |
| sorbate/water/hexylene glycol | | | | |
| Hydroxyethylcellulose | 0.50 | 0.10 | 0.10 | 0.10 |
| Ethylhexylglycerin | 0.50 | | | |
| Magnesium aluminum silicate | | 0.15 | 0.15 | 0.15 |
| Phenoxyethanol | | 0.30 | | 0.20 |
| Ammonium hydroxide | | 0.25 | 0.25 | 0.25 |
| Disodium EDTA | 0.10 | 0.10 | 0.05 | 0.05 |
| Tocopheryl acetate | | | 0.01 | 0.01 |
| Coffee Arabica (coffee) seed | | | 0.01 | 0.01 |
| extract | | | | |
| Silk amino acids | | | 0.01 | 0.01 |
| Hydrolyzed jojoba protein | | | 0.01 | 0.01 |
| PTFE | | | 0.01 | 0.01 |
| Pantothenic acid polypeptide | | | 0.01 | 0.01 |
| Simethicone | 0.08 | | | |

The compositions were prepared by grinding the pigments in a portion of the oils.

### EXAMPLE 3

An eye treatment composition was prepared as follows:

| Ingredient | % by weight |
|---|---|
| Dimethicone/vinyl dimethicone crosspolymer/water/C12-14 pareth-12 | 38.00 |
| Polydimethylsiloxane/crosslinked silicone polymer/hydrogenated coco- | 25.73 |
| glycerides/isohexadecane/glyceryl stearate/cetyl alcohol/stearyl | |
| alcohol/behenyl alcohol/cetylic acid/stearic acid/ammonium | |
| polyacryloyldimethyl taurate | |
| Water | QS |
| Silica/titanium dioxide | 5.00 |
| Simmondsia Chinensis (Jojoba) seed oil | 1.30 |
| Di C12-15 alkyl fumarate | 1.30 |
| Cetyl ricinoleate | 1.00 |
| Octyldodecyl myristate | 1.00 |
| Water/butylene glycol/lecithin/caprylyl glycol/hexylene | 1.00 |
| glycol/hydroxyethylcellulose/acetyl hexapeptide-3/hydrolyzed fish | |
| collagen/lauryldimonium hydroxypropyl hydrolyzed soy protein | |
| Water/acetyl hexapeptide-8 | 1.00 |
| Leuconostoc/radish root ferment filtrate | 1.00 |
| Pentaerythrityl tetraisostearate/silica dimethyl silylate/butylene | 1.00 |
| glycol/sodium hyaluronate | |
| Declustered water/Betula Alba extract/Saccharomyces lysate extract | 1.00 |
| Sodium acrylate/sodium acryloyldimethyl taurate copolymer/hydrogenated | 1.00 |
| polydecene/laureht-8 | |
| Glycerin | 0.90 |
| Butyrospermum Parkii (Shea butter) | 0.90 |
| Potassium cetyl phosphate | 0.90 |
| Glyceryl stearate | 0.50 |
| Whey protein | 0.50 |
| Water/butylene glycol/Zea Mays (corn) kernel extract/caprylyl | 0.50 |
| glycol/xanthan gum | |
| Sodium PCA | 0.45 |
| Water/decarboxyl caronixine HCL/butylene glycol | 0.25 |
| Caffeine | 0.20 |
| Sodium beta-sitosterol sulfate | 0.20 |
| Phytosphingosine/propylene glycol dicaprate | 0.20 |
| Glyceryl polymetharylate/PEG-8/palmitoyl oligopeptide | 0.20 |
| Siegesbeckia Orientalis extract/glycerin | 0.20 |
| Propylene glycol dicaprate/Helianthus Annus (Sunflower) seed | 0.20 |
| cake/Hordeum Vulgare (Barley) extract/Cucumis Sativus (Cucumber) fruit | |
| extract | |
| Boswellia Serrata extract | 0.10 |
| Ceteareth-20 | 0.09 |
| Pentapeptide-3 | 0.05 |
| Citric acid | 0.04 |
| Disodium EDTA | 0.036 |

The composition was prepared by combining the ingredients and mixing well to emulsify.

### EXAMPLE 4

A skin cream composition was prepared as follows:

| | % |
|---|---|
| Ingredient | by weight |
| Water | QS |
| Dimethicone/caprylyl methicone/Polysilicone-11 | 13.00 |
| Silica/titanium dioxide | 6.00 |
| Di C12-15 alkyl fumarate | 4.50 |
| Cetearyl alcohol/cetearyl glucoside | 3.00 |
| Triethylhexanoin | 3.00 |
| Glycerin | 2.00 |
| HDI/trimethylol hexyllactone crosspolymer/silica | 2.00 |
| Butylene glycol | 2.00 |
| Lauroyl lysine | 1.50 |
| Potassium cetyl phosphate | 1.50 |
| PEG-10 dimethicone | 1.20 |
| Dimethicone | 1.00 |
| Leuconostoc/Radish Root ferment filtrate | 1.00 |
| Pentaerythrityl tetraisostearate/silica dimethyl silylate/butylene | 1.00 |
| glycol/sodium hyaluronate | |
| Polyglyceryl-2 triisostearate | 1.00 |
| Glyceryl stearate | 0.70 |
| PEG-100 stearate | 0.50 |
| Cetyl alcohol | 0.50 |
| Mica/titanium dioxide | 0.50 |
| Ammonium acryloyldimethyltaurate/VP Copolymer | 0.40 |
| Sodium hyaluronate | 0.05 |
| Phenoxyethanol | 0.045 |
| Citric acid | 0.02 |
| Disodium EDTA | 0.01 |

The composition was prepared by separately combining the water and oil phase ingredients then mixing well to emulsify.

### EXAMPLE 5

Skin treatment serums are prepared as follows:

| | % by weight | |
|---|---|---|
| Ingredients | 1 | 2 |
| Water | QS | QS |
| Carthamus Tinctorius (Safflower) Oleosomes/water | 10.00 | |
| Polyglycerin-10/water | 5.00 | |
| Ammonium acryloyldimethyltaurate/VP copolymer | 1.00 | |
| Aminomethyl propanol | 0.80 | |
| Leuconostoc/Radish Root ferment | 0.80 | 2.00 |
| Phenoxyethanol | 0.20 | 0.08 |
| Methyl trimethicone | | 5.00 |
| Jojoba esters | | 3.00 |
| Cetyl ricinoleate | | 2.50 |
| PEG-100 stearate | | 2.50 |
| Dimethicone | | 3.50 |
| Trehalose | | 2.10 |
| Cetyl ethylhexanoate | | 2.00 |
| Silica | | 2.00 |
| Myristyl alcohol | | 1.00 |
| Jojoba wax/PEG-120 esters | | 1.00 |
| Hydrogenated lecithin | | 0.52 |
| Isomerized linolenic acid/linoleic acid | | 0.50 |
| Carbomer | | 0.30 |
| Fragrance | | 0.30 |
| Tribehenin | | 0.30 |
| Tromethane | | 0.27 |
| Garcinia Indica (Kokum) seed butter | | 0.10 |
| Mangifera Indica (Mango) seed butter | | 0.10 |
| Butylene glycol/Anastatica Hierochuntica | | 0.10 |
| (Rose of Jericho) extract | | |
| Glycerin/Padina Pavonica Extract | | 0.10 |
| Water/lecithin/micrococcus lysate | | 0.10 |
| Water/Arabidopsis Thaliana extract/lecithin | | 0.05 |
| Sodium hyaluronate | | 0.025 |
| Tocopheryl acetate | | 0.01 |
| Rosemary extract | | 0.01 |
| Disodium EDTA | | 0.005 |
| Sodium hyaluronate | | 0.005 |
| Anthemis Nobilis (Chamomile) Flower extract | | 0.000362 |
| Infusions of Myrtus Communis | | 0.00015 |

The compositions were prepared by combining the ingredients and mixing well.

### EXAMPLE 6

Skin care lotions with and without the radish root ferment as set forth below were comparatively tested.

| | % by weight | | |
|---|---|---|---|
| Ingredients | 1 (3) | 2 (4) | 3 (5) |
| Water | QS | QS | QS |
| Apricot kernel oil | 9.00 | 9.00 | 9.00 |
| Butylene glycol | 6.00 | 6.00 | 6.00 |
| Olive oil | 3.00 | 3.00 | 3.00 |
| Fragrance | 3.00 | 3.00 | 3.00 |
| Simmondsia Chinensis (Jojoba) seed butter | 1.50 | 1.50 | 1.50 |
| Stearic acid | 1.30 | 1.30 | 1.30 |
| Rice bran oil | 1.00 | 1.00 | 1.00 |
| Squalane | 1.00 | 1.00 | 1.00 |
| Tribehenin | 1.00 | 1.00 | 1.00 |
| Sucrose distearate/sucrose stearate | 1.00 | 1.00 | 1.00 |
| Lecuonostoc/radish root ferment | — | 1.00 | 2.00 |
| Cetearyl olivate/sorbitan olivate | 1.00 | 1.00 | 1.00 |
| Grape seed oil | 0.50 | 0.50 | 0.50 |
| Carbomer | 0.36 | 0.36 | 0.36 |
| Stearyl alcohol | 0.30 | 0.30 | 0.30 |
| Potassium hydroxide | 0.30 | 0.30 | 0.30 |
| Phenoxyethanol | 0.05 | 0.05 | 0.05 |
| Disodium EDTA | 0.05 | 0.05 | 0.05 |
| Citrus Aurantium Amara (Bitter Orange) | 0.0005 | 0.0005 | 0.0005 |
| flower extract | | | |
| Myrtus Communis - infusion | 0.0002 | 0.0002 | 0.0002 |

The samples were stored at 50° C. for one week. They were assayed for the presence of various types of bacteria initially, after 1 day and after 1 week. The results are as follows:

| Formula/ | | | Staphlococcus | | |
|---|---|---|---|---|---|
| Time | Enterobacter | Pseudomonas | Aureus | Yeast | Mold |
| 1 (Initial) | 6.5 | 6.6 | 6.2 | 6.3 | 5.4 |
| 1 (1 day) | 5.0 | 5.0 | 5.0 | 5.0 | 4.0 |
| 1 (1 week) | 6.0 | 5.0 | 3.0 | 5.0 | 4.0 |
| 2 (Initial) | 6.4 | 6.3 | 6.4 | 6.3 | 5.2 |
| 2 (1 day) | 0 | 0 | 0 | 0 | 2.3 |
| 2 (1 week) | 0 | 0 | 0 | 0 | 0 |
| 3 (Initial) | 6.5 | 6.6 | 6.2 | 6.3 | 5.4 |
| 3 (1 day) | 0 | 0 | 0 | 0 | 0 |
| 3 (1 week) | 0 | 0 | 0 | 0 | 0 |

The above results demonstrate that the radish root extract of the invention was stable in the cosmetic emulsions and, when used alone as the preservative, was able to inhibit growth of bacteria, mold, and yeast in the compositions.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A topical composition comprising an ingredient that is obtained by fermentation of Raphanus Sativus roots by the organism Leuconostoc or Lactobacillus.

2. The composition of claim 1 where the ingredient is Leuconostoc/Radish Root Ferment Filtrate.

3. The composition of 1 which is a mascara.

4. The composition of claim 3 comprising from about 0.1-95% water, from about 0.1-95% oil and from about 0.1 to 45% of a film former, and from about 0.1-30% pigments.

5. The composition of claim 4 wherein the film former is in the form of particles in aqueous dispersion.

6. The composition of claim 5 further comprising at least one volatile solvent.

7. The composition of claim 6 wherein the volatile solvent is a paraffinic hydrocarbon.

8. The composition of claim 5 further comprising at least one non-volatile oil.

9. The composition of claim 2 which is a mascara in the oil and water emulsion form comprising Leuconostoc/Radish Root Ferment Filtrate; water; at least one film former in the form of particles in aqueous dispersion; a volatile solvent selected from isododecane, isohexadecane, C9-11 isoparaffins, or mixtures thereof iron oxides; and at least one non-volatile hydrocarbon oil selected from hydrogenated or non-hydrogenated polybutene, polydecene, polyisobutene or mixtures thereof.

10. The composition of claim 2 which is a mascara in the oil and water emulsion form comprising Leuconostoc/Radish Root Ferment Filtrate; water; at least one film former; at least one fatty acid; iron oxides; and kaolin.

11. The composition of claim 2 which is a mascara in the oil and water emulsion form comprising Leuconostoc/Radish Root Ferment Filtrate; water; at least one film former; and
the reaction product of glycerin and a C6-22 fatty acid.

12. The composition of claim 11 wherein the reaction product of glycerin and the C6-22 fatty acid comprises glyceryl stearate.

13. The composition of claim 1 which is an aqueous based skin treatment composition is a skin cream or lotion in the water and oil emulsion form comprising at least one silicone, at least one botanical extract, and at least one humectant.

14. The composition of claim 13 further comprising at least one silicone elastomer.

15. The composition of claim 14 further comprising at least one peptide.

16. An oil in water emulsion skin care composition comprising from about 0.1 to 99% oil, from about 0.1-99% water, wherein the oil phase comprises at least one silicone selected from cyclomethicone, dimethicone, methyl trimethicone, PEG-10 dimethicone, or mixtures thereof; and the composition further comprises at least one organosiloxane elastomer in an amount of about 0.1 to 50%; and about 0.01-20% of at least one humectant which is butylene glycol, propylene glycol, hexylene glycol, glycerin, or mixtures thereof

17. The composition of claim 16 further comprising acetyl hexapeptide-8.

18. A aqueous based skin treatment composition comprising from about 0.1-99% water, about 0.001-15% of at least one botanical extract, and from about 0.1-45% of an aqueous phase thickening agent.

19. The composition of claim 18 wherein the aqueous phase thickening agent is a synthetic polymer comprised of acrylate repeat units.

20. The composition of claim 19 wherein the aqueous phase thickening agent is ammonium acryloyldimethyltaurate/VP copolymer.
